(19) United States
(12) Patent Application Publication
(10) Pub. No.: US 2026/0191588 A1
(43) Pub. Date: Jul. 9, 2026

(54) Systems and Methods for Hydrogel-Assisted Urinary Calculi Capture

(71) Applicants: The Board of Trustees of the Leland Stanford Junior University, Stanford, CA (US); U.S. Government as Represented by the Department of Veterans Affairs, Washington, DC (US)

(72) Inventors: Tianjia Jessie Ge, Sunnyvale, CA (US); Simon Conti, Stanford, CA (US); Joseph C. Liao, Redwood City, CA (US); Kunj Raju Sheth, San Francisco, CA (US); Shan X. Wang, Portola Valley, CA (US); Daniel Massana Roquero, Stanford, CA (US)

(73) Assignees: The Board of Trustees of the Leland Stanford Junior University, Stanford, CA (US); U.S. Government as Represented by the Department of Veterans Affairs, Washington, DC (US)

(21) Appl. No.: 18/865,312

(22) PCT Filed: May 15, 2023
(86) PCT No.: PCT/US2023/067006

§ 371 Date: Nov. 12, 2024

Related U.S. Application Data
(60) Provisional application No. 63/341,692, filed on May 13, 2022.

(51) Int. Cl. A61B 18/24; A61B 1/307; A61B 18/00; A61K 31/722; A61K 33/26

(52) U.S. Cl. CPC A61B 18/245; A61B 1/307; A61K 31/722; A61K 33/26; A61B 2018/00505; A61B 2018/00982

(57) ABSTRACT

Systems and methods for hydrogel-assisted urinary calculi capture in accordance with embodiments of the invention are illustrated. One embodiment includes a method for retrieval of urinary calculi from a urinary tract, including inserting an endoscope into the urinary tract of a patient, fragmenting urinary calculi in the urinary tract of the patient, injecting a solution of chitosan and a solution of ferumoxytol via the endoscope into the urinary tract at the location of the urinary calculi fragments to form a magnetic hydrogel, inserting a flexible magnet wire into the urinary tract of the patient via the endoscope, retrieving the flexible magnet wire from the urinary tract after the magnetic hydrogel has magnetically coupled with the flexible magnet wire such that magnetic hydrogel is pulled out of the urinary tract, wherein the magnetic hydrogel is further bound to the fragmented urinary calculi.

### CROSS-REFERENCE TO RELATED APPLICATIONS

The current application claims the benefit of and priority under 35 U.S.C. §119(e) to U.S. Provisional Ser. No. 63/341,692 entitled “Magnetic Hydrogel System to Retrieve Kidney Stone Fragments” filed May 13, 2022. The disclosure of U.S. Provisional Ser. No. 63/341,692 is hereby incorporated by reference in its entirety for all purposes. This application is further related to U.S. patent application Ser. No. 17/524,603 entitled “Magnetic Wire for Retrieval and Elimination of Calculus from the Urinary Tract”, filed Nov. 11, 2021, the disclosure of which is further incorporated by reference in its entirety.

### STATEMENT OF FEDERALLY SPONSORED RESEARCH

This invention was made with Government support under contract DK131776 awarded by the National Institutes of Health. The Government has certain rights in the invention.

### FIELD OF THE INVENTION

The invention relates to medical devices and methods for retrieval and elimination of calculus (stone fragments) from the urinary tract. More particularly, this relates to a method of coating a stone fragment with a magnetic hydrogel, and a magnetic device to retrieve the magnetically labeled stone fragments.

### BACKGROUND

Urinary calculi are solid particles in the urinary system. One type of well-known urinary calculi is the renal calculi, or kidney stones. Kidney stone disease, affects 11% of the population and accounts for >$4 billion of healthcare expenditures per year in the USA alone. Stones can be located anywhere within the urinary tract (kidney, ureter, bladder, or urethra). However, urinary calculi can form anywhere in the urinary tract. For example, when they form in the bladder, the are referred to as bladder calculi. Untreated stone disease has the potential to cause pain, infection, and loss of kidney function. Surgical management of stone disease frequently involves endoscopy of the urinary tract and lithotripsy. A common example is ureteroscopic laser lithotripsy, in which a ureteroscope is passed endoscopically through the urethra, bladder, and ureter, up to the stone, and a laser fiber is passed through the working channel of the ureteroscope to fragment the stone.

Hydrogels are a group of polymeric materials having a hydrophilic structure which renders them capable of holding large amounts of water within their three-dimensional structure.

### SUMMARY OF THE INVENTION

Systems and methods for hydrogel-assisted urinary calculi capture in accordance with embodiments of the invention are illustrated. One embodiment includes a method for retrieval of urinary calculi from a urinary tract, including inserting an endoscope into the urinary tract of a patient, fragmenting urinary calculi in the urinary tract of the patient, injecting a solution of chitosan and a solution of ferumoxytol via the endoscope into the urinary tract at the location of the urinary calculi fragments to form a magnetic hydrogel, inserting a flexible magnet wire into the urinary tract of the patient via the endoscope, retrieving the flexible magnet wire from the urinary tract after the magnetic hydrogel has magnetically coupled with the flexible magnet wire such that magnetic hydrogel is pulled out of the urinary tract, wherein the magnetic hydrogel is further bound to the fragmented urinary calculi.

In a further embodiment, the solution of chitosan has a concentration between 0.05% and 1% (w/v), prepared in 1% acetic acid.

In still another embodiment, the solution of chitosan has a concentration of 0.5%.

In a still further embodiment, the solution of ferumoxytol includes 7 nm iron oxide cores.

In yet another embodiment, the ratio of solution of chitosan to solution of ferumoxytol is 2 1.

In a yet further embodiment, the volume of solution of chitosan is 200 μL, and the volume of solution of ferumoxytol is 100 μL.

In another additional embodiment, the method captures at least 90% of calculi.

In a further additional embodiment, the method captures at least 95% of calculi.

In another embodiment again, the flexible magnet wire includes several magnets forming a magnetic assembly at the distal end of a wire, wherein the magnets are magnetically attached end-to-end and arranged with their magnetic polarities alternating in direction, wherein the magnetization direction of each of the magnets is orthogonal to the length axis of the magnetic assembly, wherein there is a ferromagnetic component along the length axis of the magnetic assembly on one side of the magnetic assembly, and wherein the magnetic field along the length axis is sufficient to attract to the surface of the flexible wire superparamagnetic nanoparticles which have bound themselves to urinary calculi fragments.

One embodiment includes a system for the retrieval of urinary calculi from a urinary tract, including a flexible magnet wire capable of being inserted into a urinary tract via an endoscope, and a solution of ferumoxytol, and a solution of chitosan, where the solution of ferumoxytol and the solution of chitosan are mixed in the urinary tract to produce a magnetic hydrogel that binds to urinary calculi fragments, and where the magnetic hydrogel is retrievable by the flexible magnet wire.

In a further embodiment again, the solution of chitosan has a concentration between 0.05% and 1% (w/v), prepared in 1% acetic acid.

In still yet another embodiment, the solution of chitosan has a concentration of 0.5%.

In a still yet further embodiment, the solution of ferumoxytol includes 7 nm iron oxide cores.

In still another additional embodiment, the ratio of solution of chitosan to solution of ferumoxytol is 2 1.

In a still further additional embodiment, the volume of solution of chitosan is 200 μL, and the volume of solution of ferumoxytol is 100 μL.

In still another embodiment again, the method captures at least 90% of calculi.

In a still further embodiment again, the method captures at least 95% of calculi.

In yet another additional embodiment, the flexible magnet wire includes several magnets forming a magnetic assembly at the distal end of a wire, wherein the magnets are magnetically attached end-to-end and arranged with their magnetic polarities alternating in direction, wherein the magnetization direction of each of the magnets is orthogonal to the length axis of the magnetic assembly, wherein there is a ferromagnetic component along the length axis of the magnetic assembly on one side of the magnetic assembly, and wherein the magnetic field along the length axis is sufficient to attract to the surface of the flexible wire superparamagnetic nanoparticles which have bound themselves to urinary calculi fragments.

One embodiment includes a composition of hydrogel for the retrieval of urinary calculi, wherein the hydrogel is formed by mixing of one part ferumoxytol solution includes 30 mg Fe/mL in mannitol solution 44 mg/mL, having a colloidal size between 17-31 nm, having a pH between 6 and 8, and where the iron particles are 7 nm oxide cores, and two parts chitosan solution having between 0.05% and 1% (w/v) concentration of chitosan prepared in 1% acetic acid, having a pH between 4.6 and 4.8.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 shows an overview according to an exemplary embodiment of the invention the magnetic device and method for retrieval of stone fragments from the urinary system. Section (a) shows stone fragments within the collecting system of a kidney. A ureteroscope is inserted up the ureter to the kidney, and functionalized nanoparticles are instilled into the collecting system, which then bind to the stone fragments. In many embodiments, a polymer is added which forms a hydrogel with the nanoparticles. Section (b) shows the magnetic device introduced through the working channel of the ureteroscope, magnetically attracting the superparamagnetically-labeled stone fragments. Section (c) shows the magnetic device and ureteroscope being withdrawn from the ureter, along with the magnetically bound stone fragments.

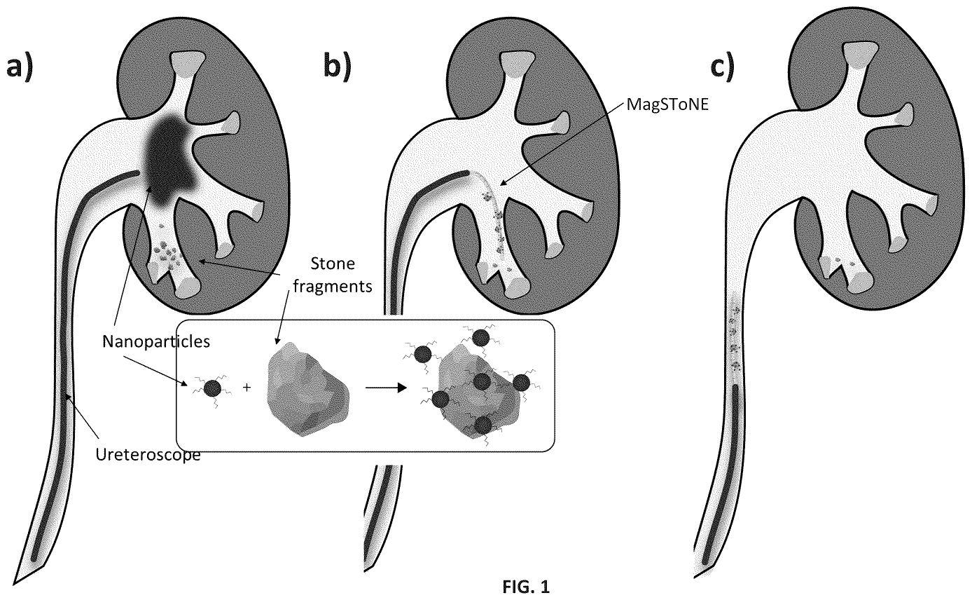

FIGS. 2A-B show according to an exemplary embodiment of the invention numerical simulations of the magnetic properties of a magnetic assembly. FIG. 2A shows a comparison of magnetic field gradient VB between different magnetic orientations (black arrows shown within the magnets point north). The alternating orientation (left image in FIG. 2A) generates high gradients and magnetic forces (‘red’ arrows shown outside the magnets, scaled logarithmically) to attract magnetic particles along the entire length, while the axial orientation (right image in FIG. 2A) only localizes gradient and force at the ends. FIG. 2B shows the gradient decays radially outward from the magnet assembly edge. Multiple points are sampled along the length of the magnet (dots), and the mean gradient is shown (solid line). The alternating polarity configuration 210 outperforms the axial configuration 220 by orders of magnitude.

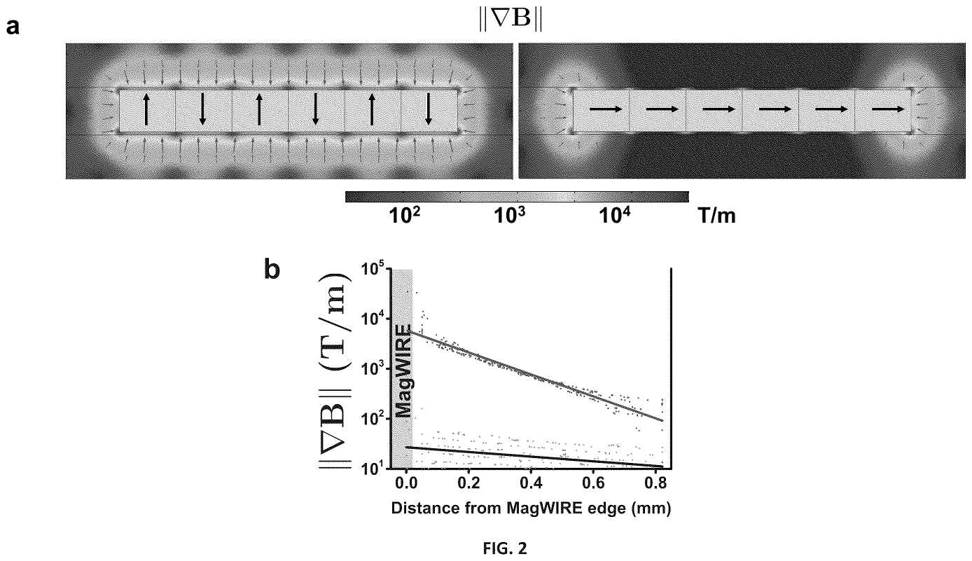

FIG. 3 shows an exploded view of the magnetic assembly according to an exemplary embodiment of the invention. A plurality of magnets (here shown two, 510, 520) are arranged within flexible sheath 530 forming a flexible assembly 500. The magnets are magnetically attached end-to-end and arranged with their magnetic polarities alternating in direction (P1 represents the north pole, while P2 represents the south pole). The magnetization direction of each of the magnets is thus orthogonal to the length axis of the assembly.

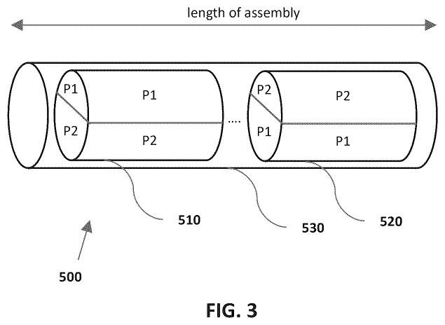

FIG. 4 shows an exploded view of the magnetic assembly according to an embodiment of the invention where the magnets are ring-shaped. A plurality of magnets (here shown two, 540, 550) are arranged with a central wire 560 to provide structural stability. There is no outer sheath. The magnets are similarly arranged end-to-end with their magnetic polarities alternating in direction (P1 and P2 are the north and south poles, respectively). The magnetization direction of each of the magnets is orthogonal to the length axis of the flexible wire.

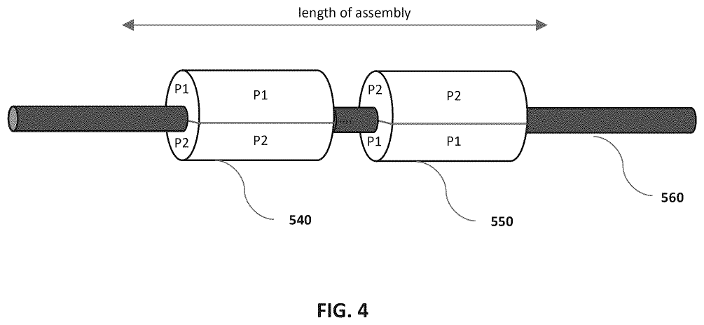

FIG. 5 shows a view of the distal tip of the magnetic device. A cross-section of the proximal catheter shows a main lumen with a ferromagnetic component 610 embedded in the catheter wall 630, and an additional lumen 620 for the deployable structure, in this case a balloon. A cross-section of the distal catheter shows how the outer wall 630 is cut away to reveal a much thinner wall 510, encasing the magnetic assembly 500. A side view of the catheter shows the entire structure.

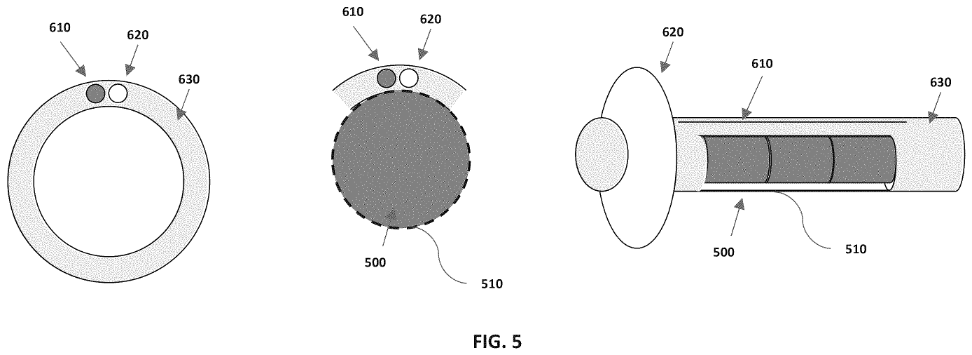

FIG. 6 shows another embodiment of the device similar to FIG. 5, where the deployable structure is instead a nitinol coil in a conical shape 640. The coil is able to retract into the lumen, but retains its coiled structure when deployed.

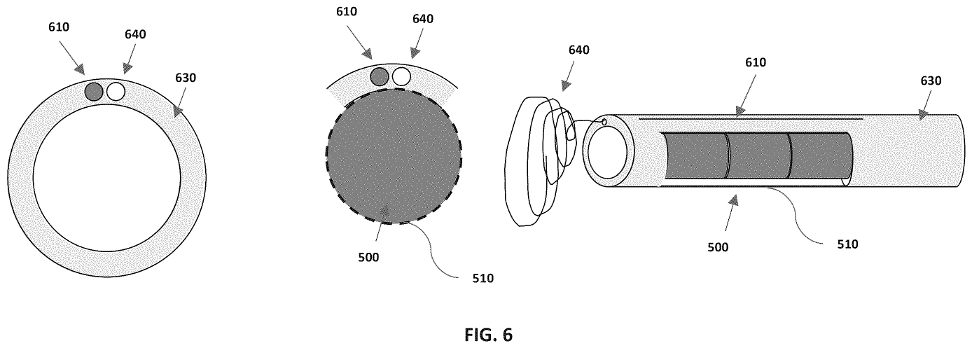

FIG. 7 shows a scanning electron microscopy image of superparamagnetic particles (3.5 μm in diameter) attached to the surface of a calcium oxalate urinary calculi fragment.

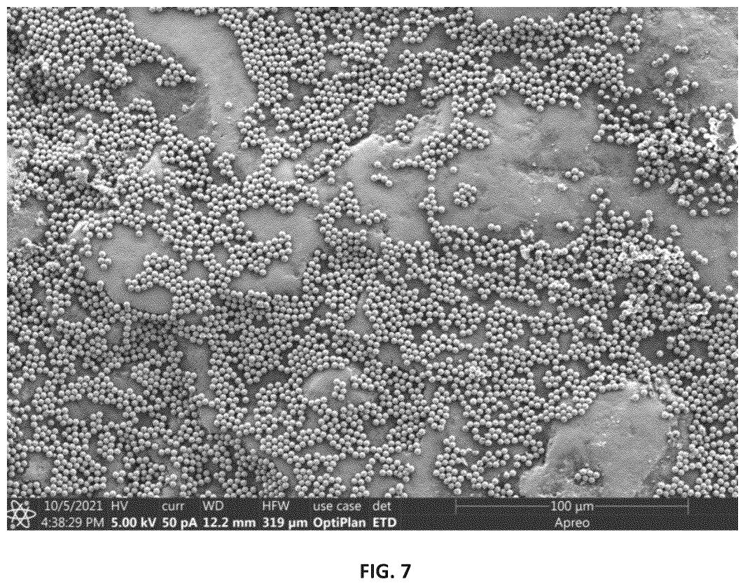

FIG. 8 shows a scanning electron microscopy image of a magnetic hydrogel composed of iron oxide superparamagnetic particles attached to the surface of a calcium oxalate urinary calculi fragment. The color overlay represents energy dispersive spectroscopy which confirms that the hydrogel seen on the right side of the image is composed of iron.

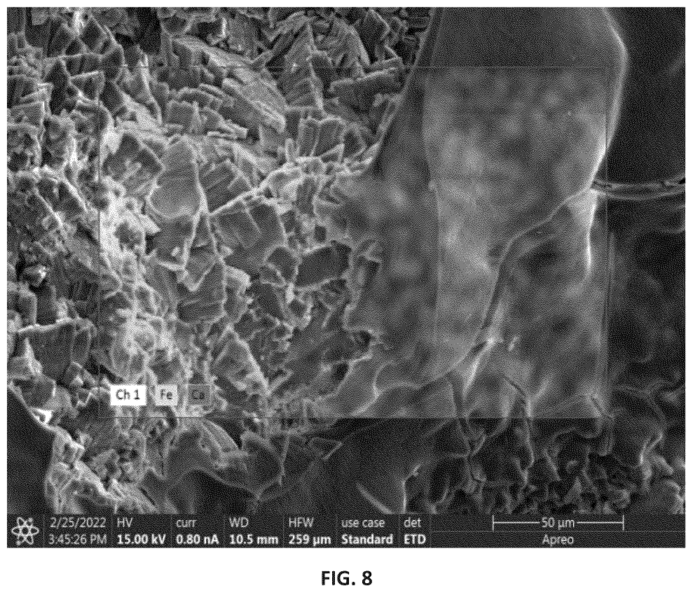

FIGS. 9A and 9B shows a simulation of magnetic field gradients for different magnetic configurations. FIG. 9a shows a colorimetric scale of the magnetic field gradient (T/m) which decays radially from the wire surface. The axial configuration has high gradients only at the ends of the wire, while the alternating configuration has high gradients across the entire length, and the Halbach configuration as high gradients across the entire length but only on one side of the magnet. FIG. 9B plots the decay of the magnetic field gradient as distance increases from the magnet surface.

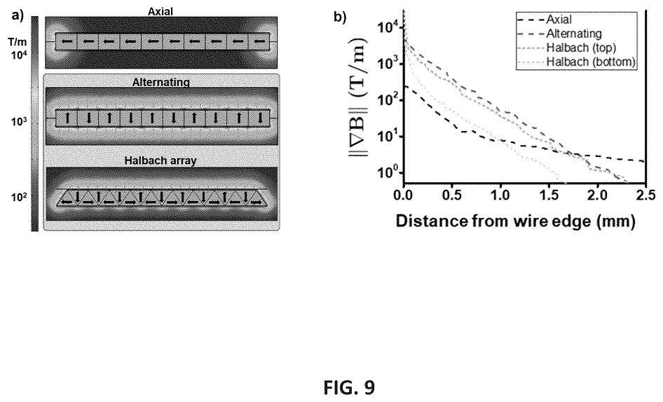

FIG. 10 shows magnetic assemblies with sheaths of different thicknesses used to capture magnetically labeled stone fragments <1 mm in size. As represented in FIG. 10, a wire with a 0.2 mm sheath will have a lower magnetic gradient at the sheath surface compared to a wire with a 0.05 mm sheath. Accordingly, wires with thinner sheaths capture a greater number of fragments, and capture larger fragments.

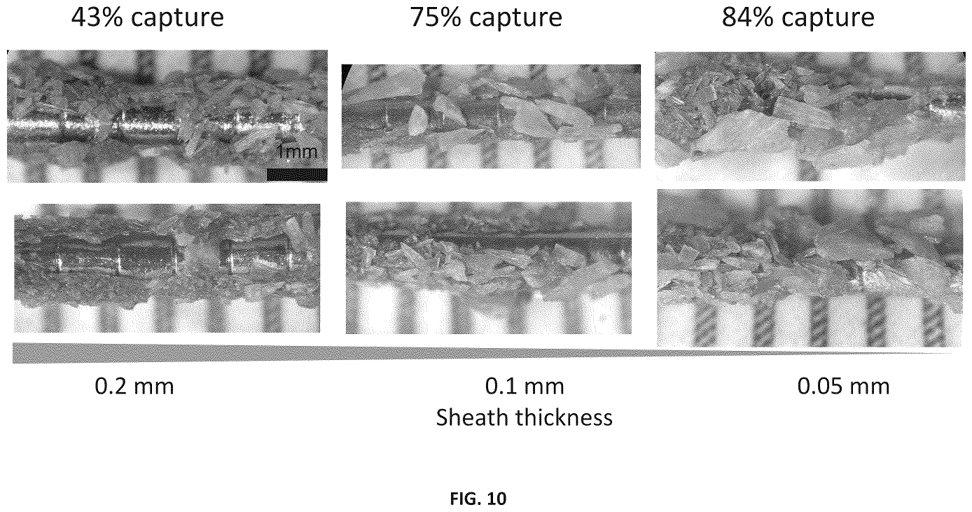

FIG. 11 is a chart demonstrating the capture efficiency of hydrogel capture compared to only ferumoxytol in accordance with an embodiment of the invention.

FIG. 12 is a flowchart for urinary calculi removal procedure using a MagSTONE device with hydrogel in accordance with an embodiment of the invention.

### DETAILED DESCRIPTION

Nephrolithiasis is a painful condition which, in the worst cases, requires invasive surgery to treat. Rendering a patient ‘stone-free’ is the best way to prevent further complications or re-interventions (stone-related events) due to residual stone fragments that can re-obstruct the ureter and kidney or grow in size. However, ‘active retrieval’ of stone fragments is associated with a significantly longer operative time due to the time spent retrieving each fragment individually. Alternatively, the stone may be fragmented into small ‘dust’ particles which are presumed to pass spontaneously during urination, but a large stone may generate such a large amount of ‘dust’ that it obscures the view of the remaining stone, causing the surgery to be stopped prematurely due to lack of visualization and necessitating a second surgery. In addition, the presumption that small dust-like fragments will pass spontaneously is not always accurate, especially in patients with limited mobility. Small fragments cannot be easily captured with current retrieval methods such as wire baskets. While larger fragments are more likely to cause stone-related events, even fragments <2 mm in size are still associated with a 20-30% rate of stone-related events. Thus, there remains a need for a method to efficiently clear urinary calculi fragments of all sizes from the body.

There have been many different configurations of wire baskets developed to increase the efficiency of stone retrieval, but these are all limited by being ill-suited to capture and retrieve multiple stone fragments at once, inability to capture small fragments, and reliance on the surgeon to guide the basket very precisely to the stone. Tan et al. (J Urol. 2012 Aug;188(2):648-5) attempted to magnetize urinary calculi fragments with paramagnetic nanoparticles, for retrieval with a magnetic tool. Their prototype magnetic tool was a single magnet measuring 8Fr (2.54 mm) in diameter, with efficacy limited by poor visualization due to the large size of the magnet, and decoupling of fragments from the magnet due to low magnetic forces.

Patent US1006453 demonstrated a magnetic wire for intravascular retrieval and enrichment (MagWIRE) which has alternating polarity magnets within a flexible sheath. The MagWIRE generates magnetic forces orders of magnitude greater than a conventional single polarity magnet. This was used in conjunction with superparamagnetic nanoparticles which could bind with disease-associated biomarkers in the bloodstream, to perform intravascular retrieval and enrichment of biomarkers from the bloodstream. The magnetic tool was slim (0.75 mm) and flexible, allowing it to be used within a small blood vessel.

Usage of the MagWIRE technology was improved upon to be able to capture urinary calculi as described in U.S. patent application Ser. No. 17/524,603, titled “Magnetic Wire for Retrieval and Elimination of Calculus from the Urinary Tract” filed on Nov. 11, 2021, which is hereby incorporated by reference in its entirety. However, there are circumstances where the improved MagWIRE will fail to attract all stone fragments, especially dust fragments. Turning now to the drawings, additional improvements upon the MagWIRE technology are described along with a hydrogel which can be used to increase stone capture between 90% and 100% of all stone fragments, including dust. These further improvements are referred to as MagSTONE (Magnetic System for Total Nephrolith Elimination). In numerous embodiments, urinary calculi are fragmented using standard uretoscopic laser lithotripsy. Then, in many embodiments, a magnetic hydrogel is formed inside the body that coats and binds the stone fragments. The gel is then removed via the MagWIRE (or via any other magnetic capture device such as, but not limited to, magnetized wire, a magnetized basket, and/or any other magnetic capture device as appropriate to the requirements of specific applications of embodiments of the invention.). Discussion of the device below is followed by a discussion of the hydrogel chemistry.

### Magstone Devices

MagSToNE devices are devices capable of introduction to the urinary tract via an endoscope that further enable the introduction of both hydrogel forming solutions and a magnetic capture device. In numerous embodiments, the endoscope is a cystoscope, ureteroscope, or nephroscope. In many embodiments, the MagSTONE device includes a magnetic assembly at the tip of a catheter or wire. A plurality of magnets is arranged to form a flexible magnetic assembly. The magnets are magnetically attached to each other, end-to-end, and arranged with their magnetic polarities alternating in direction. The magnetization direction of each of the magnets is orthogonal to the length axis of the assembly. In some embodiments, the alternating end-to-end polarities alternate 180 degrees or they could alternate with a rotation from magnet to magnet at 45-180 degree increments (for example forming a Halbach array). In a variety of embodiments, the magnets may be enclosed by a flexible sheath to ensure the structural stability of the wire. In another embodiment, the magnets may be ring-shaped with a wire running through the centers of the rings to ensure structural stability. The absence of an outer sheath in this latter embodiment can increase the maximum magnetic gradient encountered at the surface of the magnetic assembly.

There could be 5 to 1000 magnets forming the magnetic assembly with each of the magnets having a length in a range of 0.3 mm to 10 cm, with the magnetic assembly having a total length of 5 mm to 20 cm. In one example, the magnets are cylindrical magnets (rods) each having a diametric magnetization; however, the magnets do not have to be cylindrical, and could have other shapes including rings. In case of cylindrical and ring magnets they could have a diameter of 0.2 mm to 10 mm. In case of ring magnets, there can be a stainless-steel wire in the center of the rings to further augment the effective magnetic field gradient at the surface of the assembly. In one variation to this embodiment, there could additionally be non-magnetic spacers in between each magnetic unit, as a means to even further increase the magnetic field gradient along the wire.

The magnetic assembly is located at the distal tip of the device. The device is a catheter or wire with total length ranging from 50 to 200 cm. In numerous embodiments there is a ferromagnetic component embedded into the device, located on one side of the magnetic assembly. This can serve to orient the magnets and to concentrate magnetic flux towards one side of the magnet. The design of the magnetic wire with alternating magnetic polarities concentrates magnetic gradient on either side, 180 degrees from each other. However, because all ureteral access sheaths on the market have a stainless-steel coil for structural strength, the magnetic wire will attract to the side of the sheath, rendering one side inaccessible for stone retrieval as the stones on that size will shear off. Therefore, a one-sided magnet is desirable for use with current ureteral access sheaths. A ferromagnetic component on one side can orient the magnets in a uniform alignment and concentrate magnetic flux towards the opposite side of the magnet, increasing magnetic forces on the opposite side which remains exposed to attract stones and reducing forces on the ipsilateral side. In a preferred embodiment, the ferromagnetic component is a stainless-steel wire or sheet preferably measuring less than 0.01″ in diameter. In other embodiments, the material may be mumetal, or an alloy containing ferromagnetic materials such as iron, nickel, or cobalt.

In one embodiment, the MagSTONE device has a main lumen to accommodate the magnetic assembly and an additional lumen for a deployable structure (balloon, coil, or basket) which will serve to partially or fully occlude the lumen that the device is being withdrawn from. In the case of ureteroscopy, this would be the ureter or the ureteral access sheath. Macroscopic stone fragments are more likely to fall off or be knocked off the wire compared to microscopic biomarkers in the previous iteration, as they are subject to increased gravitational, shear, drag, and surface tension forces. Stones are especially prone to disengaging from the wire when they encounter air bubbles and the surface tension of the air-fluid interface, and this is especially common within the ureteral access sheath. Therefore, the device includes a deployable structure to additionally secure magnetically-attached stone fragments to the device and to retrieve fragments which have fallen off within the ureter. In one embodiment, this structure is an inflatable balloon. In another embodiment, this structure is a wire coiled in a conical shape, preferably of nitinol. The wire can be retracted into the lumen of the device where it will straighten out, and upon deployment will regain its coiled structure. In another embodiment, this structure is a wire basket. The wire coil or basket may themselves be constructed out of a magnetic material.

The proximal main lumen of the device may be occupied by a guidewire or stylet, which may be removable to modify the stiffness of the proximal catheter. Part or all of the circumference of the distal catheter wall surrounding the magnetic assembly may be removed, such that the magnetic assembly is exposed, or substantially thinned (preferably to a wall less than 0.005″, more preferably less than 0.001″), such that the magnetic assembly is encased.

In another embodiment, the MagSTONE device may have an additional lumen for a pull wire to allow for deflection of the magnetic wire tip. In one embodiment, the ferromagnetic wire serves a second function as the pull wire. In contrast to the intravenous setting which is a small confined space, the intrarenal space is much larger and more complex. Stones are often around the corner of a calyx and difficult to reach with a conventional basket. Current wires and baskets on the market do not have deflection capabilities. The ability to deflect the tip of the wire will improve its ability to capture stone fragments from difficult areas.

The MagSTONE device is dimensioned so that it can be introduced into the urinary tract. This is typically through the sheath or working channel of an endoscopic device such as a cystoscope, ureteroscope, or nephroscope. These sheaths may be as large as 30 Fr (1 cm in diameter). The device may also be introduced through the working channel of a ureteroscope, which is typically 3.6 Fr (1.2 mm). It may also be introduced through a ureteral access sheath, which is typically 11-12 Fr (3.5-3.8 mm in inner diameter). The medical device is further dimensioned to allow for removal from the sheath with stone fragments attached to it, which functionally increases the diameter of the wire depending on the size of the fragment.

The magnetic field along the length axis is sufficient to attract to the surface of the flexible wire superparamagnetic particles of the hydrogel, which bind themselves to urinary calculi which are typically composed of calcium, uric acid, struvite, or cystine. The flexible wire is a self-contained device (i.e. without the use of an external magnetic source) which could generate magnetic field gradients of 100 to 10,000 T/m. The stone fragments are then displaceable from the surface of the magnets or outer sheath with manual force, allowing for re-use of the magnetic wire after sanitization. In one embodiment, the magnets themselves can be removed from an outer sheath, thus removing the magnetic force and allowing the fragments to be more easily displaced from the sheath.

In order to enable the MagSTONE device to better capture urinary calculi, a magnetic hydrogel can be introduced into the body via the endoscopic device which binds urinary calculi fragments, including dust, which increases retrieval percentage to over 90%. The magnetic hydrogel can be formed via introduction of component solutions via a catheter placed in the working channel of the endoscope prior to introduction of the MagSTONE device.

Embodiments of the invention have several advantages. First, the flexibility and dimension of the MagSTONE device greatly resemble the guidewires which are already commonly used for ureteroscopy. The flexible tip allows it to be inserted into the urinary tract without causing trauma. The dimension makes it easily compatible with existing equipment. In addition, the removal inner stylet provides even greater control over the flexibility, and can optimize the flexibility of the ureteroscope tip while the medical device is in place. Second, the unique geometry of alternating-polarity magnetic units maintains strong magnetic field gradients (100-10,000 T/m) along the entire wire. Thus, stone fragments can be collected along the entire length of the magnetic tip of the device, rather than just at the very end of the magnet as is the case with conventional single polarity magnets, greatly increasing the yield. Additionally, the strong magnetic field can attract magnetically-labeled fragments from a distance. The predominant basket retrieval technique requires great precision on the surgeon's part to open and close the basket around a stone, which can be time-consuming and generally requires a second assistant to operate the basket. The magnetic wire could be operated by a single person, just by deflecting the tip of the ureteroscope to guide it around the kidney/ureter/bladder to attract stones.

Alternatively, in an embodiment which includes a magnetic basket configuration, the magnetic forces can simplify the process of capturing a magnetically-labeled stone in the basket. Third, while the strong magnetic field will attract a magnetically-labeled fragment to the device, if significant resistance is met upon withdrawal of the device with an attached fragment, the resisting force will remove the fragment from the magnet rather than cause damage to the urinary system. This is of utmost importance in the ureter. For example, a basket containing a stone which is too large to pass through the ureter could cause the dreaded complication of ureteral avulsion if it is withdrawn with significant resistance. In addition, it can be quite difficult to disengage a basket from the stone it is surrounding, if there is no room in the ureter to fully open the basket. The magnetic medical device will avoid this complication by only retaining the number of fragments which are small enough to be safely removed from the ureter, and easily shedding the rest. In one embodiment as described above, the magnets can be removed from the sheath to completely remove the magnetic force and disengage the device from the stone fragments. Hydrogel composition and chemistry are discussed in further detail below.

### Magnetic Hydrogel-Assisted Capture

Magnetic hydrogels as discussed herein are hydrogels that incorporate magnetic nanoparticles which can bind to urinary calculi fragments. In numerous embodiments, the gel adheres to the stone fragments via physical adsorption and electrostatic interactions. Due to the medical sensitivity of the setting, proper composition is critical both maximizing safety and maximizing retrieval. In numerous embodiments, the magnetic hydrogel is formed using two component solutions: ferumoxytol and chitosan. In many embodiments, the ferumoxytol solution contains 30 mg Fe/mL in mannitol solution 44 mg/mL, having a colloidal size ranging from 17-31 nm, and a pH ranging from 6-8. In various embodiments, the chitosan solution has between an 0.05%-1% (w/v) concentration of chitosan prepared in 1% acetic acid (pH 4.5), resulting in an overall solution pH of between 4.6-4.8. In numerous embodiments, the chitosan solution has a 0.5% concentration. When the ferumoxytol and chitosan solutions mix, they create a magnetic hydrogel appropriate for safe and complete capture of urinary calculi via a MagSTONE device.

In some embodiments, glycerol can be added to the chitosan solution with a final concentration of 10% (v/v) to improve injectability and setting time. This is because the ferumoxytol solution is a dense liquid compared to the chitosan solution, and immediately settles during injection. Glycerol increase the density of the chitosan solution, increasing the setting time and enhancing hydrogel formation near the stone surface with the already settled ferumoxytol solution. While glycerol does not participate directly in gel formation, it can improve fragment removal.

The amount of solution to be injected is dependent upon the stone burden, size ranges, location, and distribution of the stone fragments. In most cases, a single dose of 100 μL of ferumoxytol solution and 200 μL of chitosan solution is sufficient to achieve stone-free status. For large burdens, additional injection cycles may be required. For larger or smaller amounts, a 1:2 ratio of ferumoxytol solution to chitosan solution can be used. However, as total volume increases, gel formation may increase but not all nanoparticles tend to incorporate, and therefore validation of high-volume injections is advised.

Typically, gelation time occurs within 60-120 seconds when the two solutions mix. In many gel formation scenarios, gelation is achieved via mixing. However, in the kidney, it is difficult to mix without diluting using irrigant or using excessive amounts of material. In many embodiments, the two solutions are injected substantially simultaneously via a dual lumen catheter so that they are more likely to encounter each other on the surface of the stone fragments. In some embodiments, the catheter is designed such that turbulent flow is produced at the injection site when both solutions are injected in order to create mixing.

With respect to the safety of the solutions, chitosan specifically can provide benefits. Polycations are generally not used in biomedical applications as they are relatively toxic to tissue. They tend to bind to negatively charged cell walls, cause hemolysis, and agglutination. Polyanions are considered less toxic, and therefore the majority of superparamagnetic iron oxide nanoparticles (SPIONs) for intravenous use have anionic coatings rather than cationic coatings. Chitosan is generally regarded as a ‘non-toxic’ polycation but one of the reported toxicities is desquamation of the urothelial lining. However, the effect in this case does not appear to be clinically significant, and agglutination of red blood cells as a side effect of chitosan use can help clot red blood cells and improve visibility in bloody urine/saline during the procedure. Further, its cytotoxicity to bacteria may reduce injection and sepsis rates after surgery.

Ferumoxytol is additionally beneficial as the 7 nm iron oxide cores are small enough to not occlude capillaries if there is accidental introduction to the blood stream. Further, ferumoxytol is advantages as the small cores do not magnetically separate due to Brownian motion overwhelming the attractive magnetic forces. This makes ferumoxytol useful component of the hydrogel.

In many embodiments, additional components can be added to the hydrogel to create various effects. For example, in numerous embodiments, additional magnetic nanoparticles can be added into the chitosan solution in order to increase the total amount of magnetic particles in the hydrogel. In various embodiments, an enzyme (e.g. lysozyme) can be added to either or both solutions that speed up biodegradation of the hydrogel.

While ferumoxytol and chitosan solutions are described above, other hydrogels can be used without departing from the scope or spirit of the invention. In numerous embodiments, the superparamagnetic particles in the hydrogel can range in size from 5 nm to 5 microns. They may be functionalized with chemical groups that will bind to stone fragments composed of calcium, uric acid, struvite, or cystine, thus coating the surface of the fragment and rendering it magnetizable. In one embodiment, these chemical groups may be carboxylic acid or phosphate groups which would bind via ionic interactions with exposed cations on the surface of the stone fragment, or amine groups which would similarly bind with exposed anions. These may be groups directly functionalized to the surface of the particle, or comprising part of a larger molecule such as carboxylic acid groups in polyaspartic acid or polyacrylic acid.

In another embodiment, the particle solution is mixed with a polymer to facilitate flocculation or gelation of the particles to increase the number of particles that are bound to the stone. Macroscopic stone fragments also have a lower surface area-to-volume ratio and greater density compared to microscopic biomarkers. Thus, the attractive magnetic force of a stone fragment with a surface coating of superparamagnetic particles will eventually be outweighed by the countering gravitational force, which is a consideration not applicable to microscopic biomarkers. Thus, the method may require modifications to increase the number of particles labeling a fragment to generate a magnetophoretic force which can overcome gravitational force. In one embodiment, the gelation occurs via ionic interaction. Preferably, particles are negatively charged with a carboxylic acid coating, and the polymer is positively charged. In this embodiment the polymer is preferably chitosan, preferably of a low molecular weight, at a pH between 3.5 and 5.5, and a concentration between 0.05% and 2% w/v. In another embodiment, the particles are positively charged with an amine coating, and the polymer is negatively charged. In another embodiment, it is the polymer which is functionalized with the aforementioned chemical groups that bind to the stone fragment, and the particles are functionalized with groups that cross-link the polymer. The component which binds to the stone is introduced first to provide a base coating and high concentration on or near the stone. The second component is introduced in a separate step to promote flocculation or gelation. Ideally, by concentrating the initial coating on the stone via functional groups that bind to the stone, gelation is concentrated on the stone surface, and gelation that occurs elsewhere results in a material with weak cross-linking due to dilution. Thus, excess gel does not form a solid and is easily irrigated away. A procedure for using hydrogels in conjunction with the MagSTONE device are discussed below.

### Hydrogel-assisted Capture Procedure

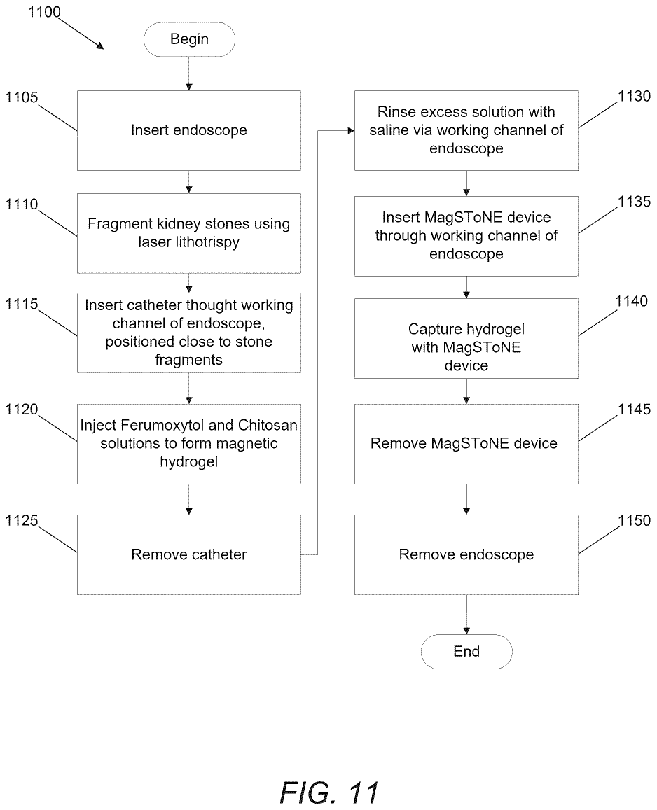

An advantage of hydrogel-assisted urinary calculi capture procedures is that they can take place in conjunction with conventional urinary calculi fragmentation techniques, and can be performed using standard endoscopic devices. Aside from hydrogel components and a MagSTONE device, no other modifications to an operating theater are required. Turning now to FIG. 11, a flow chart for a hydrogel-assisted capture procedure in accordance with an embodiment of the invention is illustrated.

Procedure 1100 includes inserting (1105) an endoscope into the patient's renal system. In numerous embodiments, the endoscope is a cystoscope, ureteroscope, or nephroscope as appropriate to the patient. Urinary calculi in the patient are fragmented (1110) using laser lithotripsy. This creates urinary calculi fragments, including dust. A catheter is inserted (1115) through the working channel of the endoscope and positioned close to the fragments. Ferumoxytol and chitosan solutions are injected (1120) via the catheter to form a magnetic hydrogel that binds the fragments. The catheter is removed (1125) and excess solution is rinsed (1130) saline delivered via the working channel of the endoscope. The MagSTONE device is inserted (1135) via the working channel of the endoscope and used to capture (1140) the hydrogel coated fragments. The MagSTONE device is then removed (1145) from the body, bringing the hydrogel and fragments with it. The endoscope is removed (1150) and the patient can be monitored during recovery. As can be readily appreciated, additional steps may be required for the safety of a given patient for any number of different medical reasons. Further, some patients may not need rinsing, or may alternatively require additional cycles of hydrogel injection and removal to completely capture all fragments in cases of significant stone burden.

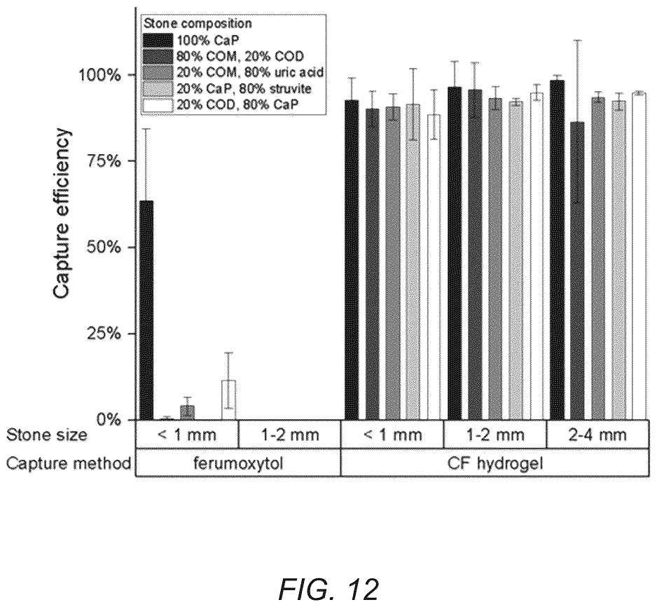

The hydrogel-assisted capture procedure described herein has been empirically shown achieve retrieval efficiency of above 90%, inclusive of both dust and larger calculi fragments of a variety of compositions. Turning now to FIG. 12, a chart showing hydrogel-assisted capture efficiency as compared to using superparamagnetic particles (e.g. ferumoxytol) alone. As can be seen, stones of various compositions and sizes were all captured with very high rates of efficiency as compared to superparamagnetic particles alone which are only reasonably effective for small particles.

Although specific devices and methods for hydrogel-assisted urinary calculi capture are discussed above, many different modifications can be implemented in accordance with many different embodiments of the invention. For example, additional lumens or lumen placement may be modified as appropriate to the requirements of specific applications of embodiments of the invention. Further, additional hydrogel compositions can be considered for use with a MagSTONE device. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for retrieval of urinary calculi from a urinary tract, comprising:
inserting an endoscope into the urinary tract of a patient;
fragmenting urinary calculi in the urinary tract of the patient;
injecting a solution of chitosan and a solution of ferumoxytol via the endoscope into the urinary tract at the location of the urinary calculi fragments to form a magnetic hydrogel;
inserting a flexible magnet wire into the urinary tract of the patient via the endoscope; and
retrieving the flexible magnet wire from the urinary tract after the magnetic hydrogel has magnetically coupled with the flexible magnet wire such that magnetic hydrogel is pulled out of the urinary tract, wherein the magnetic hydrogel is further bound to the fragmented urinary calculi.

2. The method of claim 1, wherein the solution of chitosan has a concentration between 0.05% and 1% (w/v), prepared in 1% acetic acid.

3. The method of claim 1, wherein the solution of chitosan has a concentration of 0.5%.

4. The method of claim 1, wherein the solution of ferumoxytol comprises 7 nm iron oxide cores.

5. The method of claim 1, wherein the ratio of solution of chitosan to solution of ferumoxytol is 2:1.

6. The method of claim 5, wherein the volume of solution of chitosan is 200 μL, and the volume of solution of ferumoxytol is 100 μL.

7. The method of claim 1, wherein the method captures at least 90% of calculi.

8. The method of claim 1, wherein the method captures at least 95% of calculi.

9. The method of claim 1, wherein the flexible magnet wire comprises:
a plurality of magnets forming a magnetic assembly at the distal end of a wire, wherein the magnets are magnetically attached end-to-end and arranged with their magnetic polarities alternating in direction;
wherein the magnetization direction of each of the magnets is orthogonal to the length axis of the magnetic assembly;
wherein there is a ferromagnetic component along the length axis of the magnetic assembly on one side of the magnetic assembly; and
wherein the magnetic field along the length axis is sufficient to attract to the surface of the flexible wire superparamagnetic nanoparticles which have bound themselves to urinary calculi fragments.

10. A system for the retrieval of urinary calculi from a urinary tract, comprising:
a flexible magnet wire capable of being inserted into a urinary tract via an endoscope;
a solution of ferumoxytol; and
a solution of chitosan;
where the solution of ferumoxytol and the solution of chitosan are mixed in the urinary tract to produce a magnetic hydrogel that binds to urinary calculi fragments; and
where the magnetic hydrogel is retrievable by the flexible magnet wire 12. The system of claim 10, wherein the solution of chitosan has a concentration between 0.05% and 1% (w/v), prepared in 1% acetic acid.

13. The system of claim 10, wherein the solution of chitosan has a concentration of 0.5%.

14. The system of claim 10, wherein the solution of ferumoxytol comprises 7 nm iron oxide cores.

15. The system of claim 10, wherein the ratio of solution of chitosan to solution of ferumoxytol is 2:1.

16. The system of claim 10, wherein the volume of solution of chitosan is 200 μL, and the volume of solution of ferumoxytol is 100 μL.

17. The system of claim 10, wherein the method captures at least 90% of calculi.

18. The system of claim 10, wherein the method captures at least 95% of calculi.

19. The system of claim 10, wherein the flexible magnet wire comprises:
a plurality of magnets forming a magnetic assembly at the distal end of a wire, wherein the magnets are magnetically attached end-to-end and arranged with their magnetic polarities alternating in direction;
wherein the magnetization direction of each of the magnets is orthogonal to the length axis of the magnetic assembly;
wherein there is a ferromagnetic component along the length axis of the magnetic assembly on one side of the magnetic assembly; and
wherein the magnetic field along the length axis is sufficient to attract to the surface of the flexible wire superparamagnetic nanoparticles which have bound themselves to urinary calculi fragments.

20. A composition of hydrogel for the retrieval of urinary calculi, wherein the hydrogel is formed by mixing of:
one part ferumoxytol solution comprising 30 mg Fe/mL in mannitol solution 44 mg/mL, having a colloidal size between 17-31 nm, having a pH between 6 and 8, and where the iron particles are 7 nm oxide cores; and
two parts chitosan solution having between 0.05% and 1% (w/v) concentration of chitosan prepared in 1% acetic acid, having a pH between 4.6 and 4.8.
